(19) United States
(12) Patent Application Publication
(10) Pub. No.: US 2006/0234383 A1
(43) Pub. Date: Oct. 19, 2006

(54) Tissue assessment

(76) Inventor: Nigel Gough, Rhiwbina (GB)

Correspondence Address: Dewitt Ross & Stevens;Intellectual Property Department, Firstar Financial Centre, Suite 401 8000 Excelsior Drive, Madison, WI, 53717-1914, US

(21) Appl. No.: 10/546,178

(22) PCT Filed: Feb. 19, 2004
(86) PCT No.: PCT/GB04/00674

§ 371 Date: Jan. 3, 2006

(30) Foreign Application Priority Data
Feb. 19, 2003 (GB) 0303797.5

(51) Int. Cl. G01N 33/48

(52) U.S. Cl. 436/63; 436/164

(57) ABSTRACT

The tissue assessment device consists of a fluid pressure system (1, 2, 11); an optical system (3, 5, 6); and a micro-controller (13). Compression of bladder (11) by actuator (12) displaces the fluid within the bladder 11 into chamber (2) causing diaphragm (1) to inflate and apply pressure onto the tissue surface in order to initiate a blanch. The diaphragm (1) is deflated after a predetermined time, either by releasing the actuator (12) or opening exhaust valve (8). Throughout the blanching, the optical system (3, 5, 6) illuminates the blanch area and the returned light data is collected at regular intervals for several wavelengths from the point when the blanch is initiated, throughout the blanching and a predetermined time thereafter during the recovery phase. The micro-controller controls the process and analyses the returned signals to provide assessment of the tissue surface area. The invention provides a simple low skill tissue assessment device that is highly reliable.

The present invention relates to a tissue assessment device and method. More particularly, the present invention relates to a device and method for the assessment of microvasculature damage in the tissue. The invention is applicable to conditions that affect the haemodynamics of the microvasculature such as ischaemia, early stage pressure damage, white finger syndrome (WFS), Reynaud's syndrome and diabetic peripheral neuropathy.

Skin evaluation devices are known that use reflectance spectroscopy to assess skin perfusion. A known device uses a sliding blanch technique to push blood out of the skin immediately below an optical sensor and a photo detector. The optical sensor delivers light to the skin and measures the intensity of light reflected back. The sliding blanch technique involves the operator holding one end of a probe against the skin and either pushing or pulling it along the surface of the skin a ridge on the probe causing the blanching. Unfortunately, it has been found that this device is acceptable only with a skilled practitioner thereby restricting its use

An object of the present invention is to seek improvements.

Accordingly, one aspect of the present invention comprises a tissue microvasculature assessment method including the steps of pushing blood out of a tissue area for a period of time by means of applying fluid pressure, allowing the blood to re-enter the area by removing the fluid pressure, illuminating the area throughout the procedure, collecting the returned light and analysing the results to provide assessment of the microvasculature damage at the area.

In another aspect of the present invention, there is provided a tissue microvasculature assessment device comprising a means to apply fluid pressure onto a tissue surface area to initiate a blanch and removed thereafter, an optical system to deliver and collect light to and from the area, the values of returned light analysed to provide discrimination of microvasculature damage in the area. Therefore, the application of fluid pressure onto the tissue surface pushes blood out of the area of microvasculature in the upper layers of tissue directly below the optical measuring system wherein the optical system return light values provide information of the variation with time of blood as it refills the volume previously evacuated. This information is input into a classification algorithm trained using reference data and analysed to provide discrimination of microvasculature damage.

In a preferred embodiment, the means of applying fluid pressure comprises a diaphragm inflated by air to apply pressure onto the tissue surface and deflated to release the applied pressure. The application of pressure by the inflated diaphragm is gentle onto the tissue surface and does not cause pain or discomfort. Preferably, the application of pressure on the tissue surface is constant for a predetermined time. Alternatively, the pressure applied can be pulsed and/or of varying pressure. More preferably, the pressure applied is rapidly released to allow for blood reflow into the area.

Preferably, the diaphragm is transparent so that light transmission and return paths can pass through the diaphragm.

Preferably, when the application of pressure is released the diaphragm relaxes and moulds itself to the contour of the tissue surface preventing extraneous light from entering the optical sensor and reducing measurement errors. Also provided, is a diaphragm for use with such a tissue microvasculature assessment device, the diaphragm comprising a transparent flexible membrane adapted to be fitted over the end of the tissue microvasculature assessment device.

In a further aspect of the present invention, there is provided a tissue microvasculature assessment device comprising a means to apply fluid pressure onto a tissue surface test site to initiate a blanch and removed thereafter, an optical system to deliver and collect light to and from the test site during the initiation of the blanch, upon cessation of the blanch, and for a period of time after, the values of returned light analysed to provide assessment of limb ischaemia. Preferably, the device can also be used to provide assessment of diabetic peripheral neuropathy.

An embodiment of the present invention is described below, by way of example only, with reference to the accompanying drawings in which:

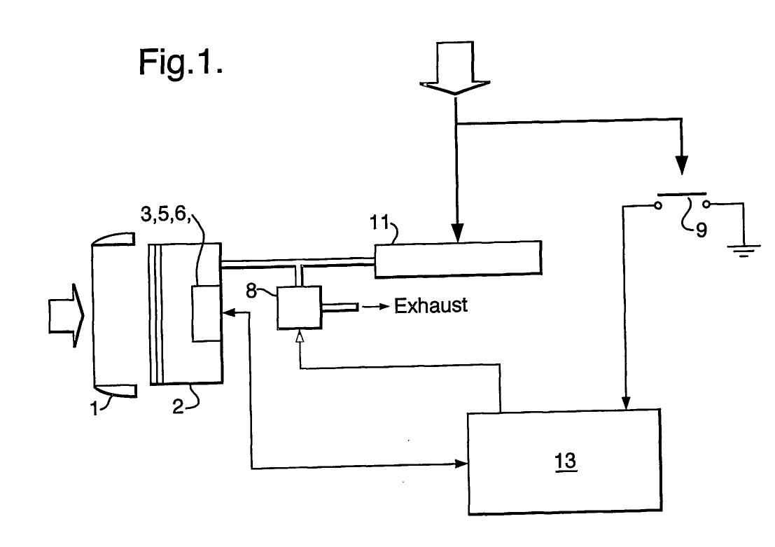

FIG. 1 is a system diagram of a tissue microvasculature assessment device according to the invention;

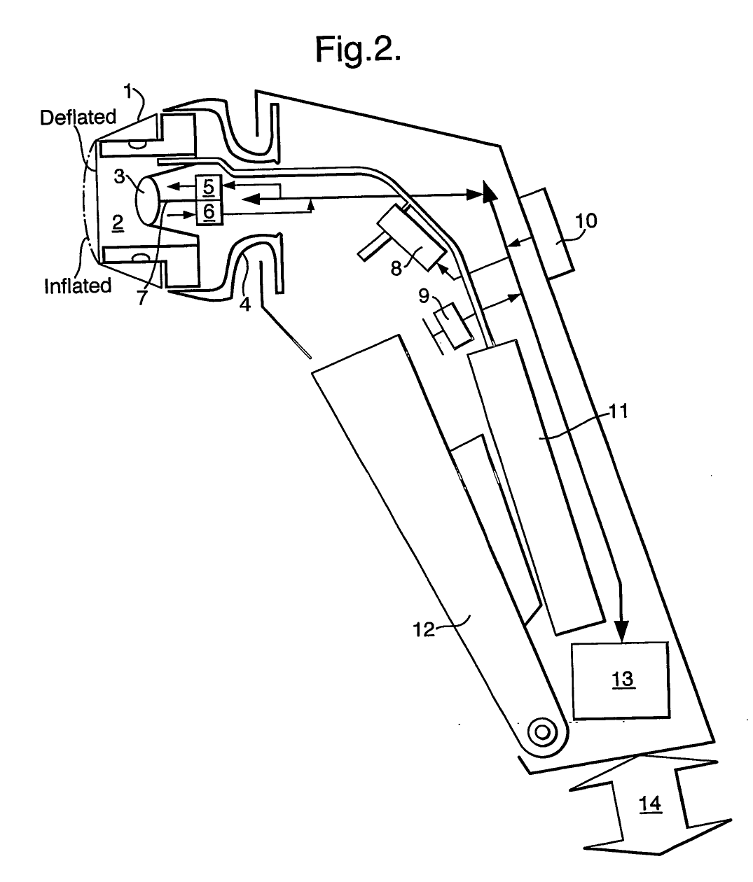

FIG. 2 is a schematic diagram of a preferred embodiment of the tissue microvasculature assessment device;

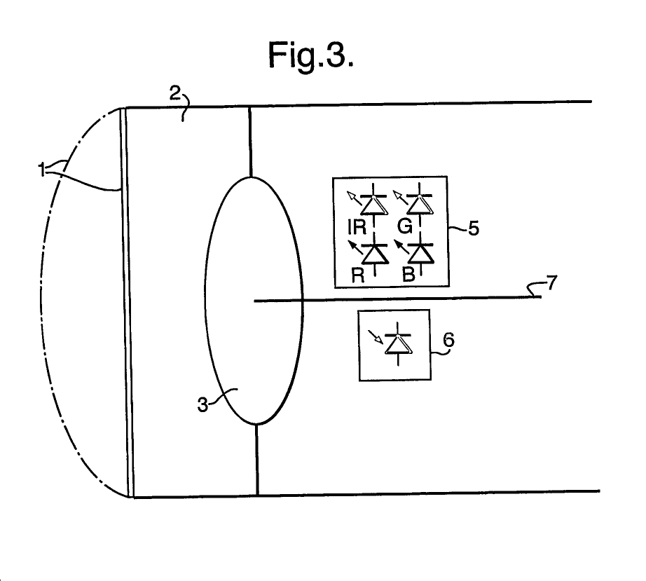

FIG. 3 shows the optical system of the device in FIG. 2; and

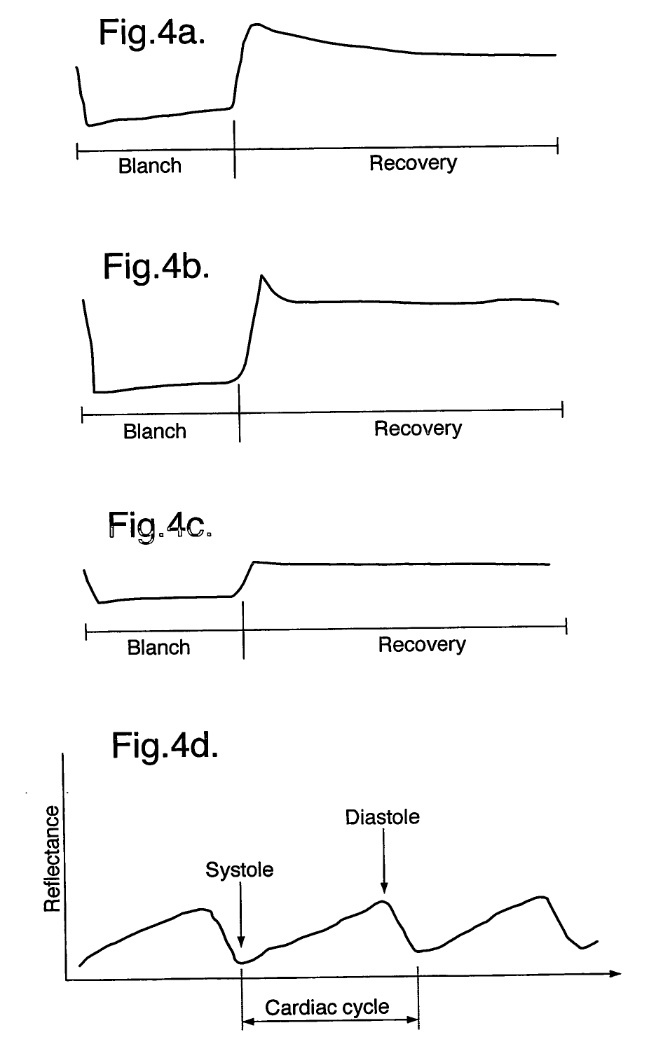

FIG. 4 shows the one set of results obtained from the device.

Referring to FIG. 1, the tissue microvasculature assessment system consists of a fluid pressure system 1,2,11 to produce a blanch, an actuator 12 to actuate the blanch, an optical system to illuminate the blanch and receive the reflected signals and a micro-controller 13 to control the blanch process, the optical process and take measurements.

S As shown in FIG. 2, the preferred embodiment has a bladder 11 with stiff walls constituting a pump, an open ended chamber 2 housing the optical system 3, 5, 6 and a transparent, flexible diaphragm 1 stretched over the open end of chamber 2, all comprising the fluid pressure system to apply a blanch force to the tissue surface. The diaphragm 1 can also be inflated by any other suitable means, such as a small pump activated by a switch. The switch could be optical or mechanical or similar.

The diaphragm 1 is fitted over the end of the chamber 2 thereby sealing the chamber 2 and causing the whole fluid pressure system to be sealed. Compression of bladder 11 by actuator 12 displaces the fluid within the bladder 11 into chamber 2 causing diaphragm 1 to inflate to initiate the blanch onto the tissue surface. To remove the applied pressure, the diaphragm 1 is deflated either by releasing the actuator 12 or opening an exhaust valve 8. The exhaust valve 8 serves to balance the internal pressure with the external atmospheric pressure before making a measurement, and to rapidly deflate the diaphragm 1 after a timed blanch force has been applied before making the refill measurement.

The micro-controller 13 communicates with the external systems, for example a display, controls the analogue to digital conversion of the received reflectance signal from the optical sensor 3,5,6; controls the light sources in the optical sensor 3,5,6; and controls the exhaust valve 8. The optical sensor comprises a light source 5 including infrared, red, green and blue LEDs and a photo detector 6. The optical system typically, but not exclusively, consists of several LEDs the wavelengths of which are chosen to suit the application. A typical combination may be infrared, red, green and blue. For the measurement of returned light a photo-detector 6 is used. The light to and from these components, may be coupled to the tissue surface by a fibre optic bundle or directly through a lens 3 (see FIG. 3). The light source 5 and the photo-detector 6 are separated by a baffle 7 to prevent light from the light source 5 coupling directly with the photo-detector 6. The diaphragm 1 is of translucent silicon allowing light to pass through to the target tissue surface area.

In use, diaphragm 1 is attached to the probe head 2. and the face of the diaphragm 1 held against the tissue surface. A switch 10 initiates the calibration process. When calibration is complete, an audible or visual prompt informs the operator to squeeze the actuator 12. Actuator 12 compresses bladder 11 to displace the fluid, in this case air, within the bladder 11 into chamber 2 causing diaphragm 1 to inflate thus applying a force to the tissue surface to initiate a blanch. The blanch is timed by micro-controller 13, and after a predetermined time, the exhaust valve 8 is opened to rapidly deflate the diaphragm 1 to remove the blanching force. Throughout the blanching, the optical system 3,5,6 illuminates the blanch area and the returned light data is collected at regular intervals for each wavelength from the point when the blanch is initiated, throughout the blanching and a predetermined time thereafter. Although the preferred embodiment shows the arrangement as shown in FIG. 2, other arrangements of the invention are possible in order to provide the tissue assessment device.

Typical blanch signals are shown in FIG. 4. FIG. 4a shows a typical normal signal with a slow refill curve during the recovery period. FIG. 4b shows a refill curve with fast refill curve during the recovery phase. FIG. 4c shows the curve typical of a non-blanching erythema, that is when the tissue microvasculature is damaged and there is no dynamic recovery curve since none of the blood has been blanched from the target tissue area. Furthermore, the device detects microvasculature pulsation (FIG. 4d) associated with inflammation and hyperaemic response in those areas of tissue which, unlike the soles of the feet and the palms of the hands, are not normally densely vascularised and therefore where a pulse is normally not seen. This pulsation in conjunction with the dynamic refill signal provides a powerful classification data set to train a classification algorithm.

Such classification algorithms include Artificial Neural Networks (ANN) or polynomial curve fitting algorithms. The output of these classification algorithms can be communicated to the operator either by a simple display in the hand unit or on a host computer linked by the bi-directional data link 14. For example, in the case of pressure area tissue damage the information relayed to the operator is in the form of 3 categories, 1 NORMAL (non red area blanchable with normal refill curve), 2 BLANCHING HYPERAEMIA (red area which may not be seen in pigmented skin, that is blanchable with a shorter refill curve), 3 NONBLANCHING HYPERAEMIA (persistent red area which may not be seen in pigmented skin, that does not show a visible blanch.) Also, any blood supply manifest by a pulsatile component implying a resolvable condition can also be presented on the screen to the operator. Absence of such a pulsatile component could indicate severe damage.

Alternatively, where the assessment device is used for the assessment of critical limb ischaemia, the output takes the form of 2 categories such as, 1 SUFFICIENT FLOW, 2 INSUFFICIENT FLOW.

Further, it is also possible to analyse the data collected to determine physiologically related indices, for example, calculating the polynomial coefficients of curves fitted to the refill data or ratios of systolic and diastolic points in the pulsatile waveform or gradiants of the pre and post systolic peaks/troughs pulse waveform to provide an indication of tissue state.

Tissue assessment using the device according to the invention is easy to use without requiring any particular training while at the same time enhancing measurement repeatability. The invention provides a simple low skill tissue assessment device that is highly reliable. The device simulates the method used by Tissue Viability Specialists blanching the skin with the tip of the finger but provides a consistent blanch and a repeatable, measurable diagnosis regardless of patient skin colour.

Moreover, the measurements are taken from the same area of tissue that the device is calibrated on with the area of measurement larger than previous arrangements thereby giving stronger signals and the pressure applied for blanching is repeatable. The term tissue used throughout the description covers skin and any other tissues including internal body membranes and tissue surfaces exposed by surgical means, where such assessment is beneficial.

1-12. (canceled)

13. A tissue assessment method comprising the steps of:
a. applying fluid pressure to a tissue surface area to push blood out of the area;
b. removing the fluid pressure to allow blood to re-enter the area;
c. illuminating the area during the foregoing steps,
d. collecting the returned light, and
e. analyzing the results to provide assessment of the microvasculature damage at the area.

14. The tissue assessment method of claim 13 wherein the pressure applied onto the area is constant for a predetermined time.

15. The tissue assessment method of claim 13 wherein the pressure applied onto the area is pulsed.

16. The tissue assessment method of claim 13 wherein varying pressure is applied onto the area.

17. The tissue assessment method of claim 13 wherein the pressure applied is rapidly released to allow blood reflow into the area.

18. The tissue assessment method of claim 13 wherein an inflatable diaphragm applies the fluid pressure to the tissue surface area.

19. The tissue assessment method of claim 18 wherein:
a. the diaphragm is transparent, and
b. the optical system collects the returned light through the diaphragm.

20. The tissue assessment method of claim 18 wherein the diaphragm molds itself to the contour of the tissue area when fluid pressure is removed.

21. The tissue assessment method of claim 13 used to assess limb ischaemia.

22. The tissue assessment method of claim 13 used to assess diabetic peripheral neuropathy.

23. A tissue assessment device comprising:
a. means for applying fluid pressure onto a tissue surface area to initiate a blanch,
b. an optical system to deliver and collect light to and from the area, wherein the values of collected light may be analyzed to provide a measure of microvasculature damage at the tissue area.

24. The tissue assessment device of claim 23 wherein the means for applying fluid pressure comprises an inflatable diaphragm.

25. The tissue assessment device of claim 24 wherein:
a. the diaphragm is transparent, and
b. the optical system delivers and collects light through the diaphragm.

26. The tissue assessment device of claim 23 further comprising a rigid housing:
a. wherein at least a portion of the optical system is located, and
b. having an end whereupon the means for applying fluid pressure is situated.

27. The tissue assessment device of claim 26 wherein the means for applying fluid pressure is defined by a flexible diaphragm removably fittable about the end of the housing.

28. The tissue assessment device of claim 27 wherein two or more light sources:
a. are within the housing and are oriented to emit light through the diaphragm, and
b. emit light having different mean wavelengths.

29. The tissue assessment device of claim 26 wherein:
a. the housing is defined by a probe sized and configured to be held and manipulated by a single hand, and
b. the housing bears an actuator thereon which actuates the fluid supply, the actuator being configured to be actuated by any hand holding the housing.

30. A tissue assessment device comprising:
a. a housing terminating in a flexible diaphragm, with a chamber being provided in the housing behind the diaphragm;
b. an actuatable fluid supply in communication with the chamber, wherein the fluid supply supplies fluid to and from the chamber to flex the diaphragm;
b. a light source within the housing, wherein the light source emits light through the diaphragm;
c. a photodetector within the housing, wherein the photodetector detects any light returned by a tissue surface area from the light source.

31. The tissue assessment device of claim 30 wherein:
a. the housing is defined by a probe sized and configured to be held and manipulated by a single hand, and
b. the housing bears an actuator thereon which actuates the fluid supply, the actuator being configured to be actuated by any hand holding the housing.

32. The tissue assessment device of claim 30 wherein the diaphragm is removably fittable about an end of the housing

33. The tissue assessment device of claim 30 wherein two or more light sources:
a. are within the housing and are oriented to emit light through the diaphragm, and
b. emit light having different mean wavelengths.
